(19) United States
(12) Patent Application Publication
(10) Pub. No.: US 2018/0361377 A1
(43) Pub. Date: Dec. 20, 2018

(54) SAMPLE MANUFACTURING METHOD, SAMPLE MANUFACTURING KIT, OBSERVATION METHOD, AND OBSERVATION DEVICE

(71) Applicant: OLYMPUS CORPORATION, Tokyo (JP)

(72) Inventors: Brendan BRINKMAN, Hopkinton, MA (US); Yoshihiro SHIMADA, Kanagawa (JP)

(73) Assignee: OLYMPUS CORPORATION, Tokyo (JP)

(21) Appl. No.: 15/985,022

(22) Filed: May 21, 2018

(30) Foreign Application Priority Data
Jun. 20, 2017 (JP) 2017-120214

(51) Int. Cl. B01L 3/00; C12M 1/12; G01N 35/10; G01N 21/01; G01N 21/88

(52) U.S. Cl. CPC B01L 3/5088; C12M 25/01; C12M 25/16; G01N 35/10; G01N 21/01; G01N 2021/035; B01L 2200/025; B01L 2200/0647; B01L 2300/069; G01N 2035/1046; G01N 2021/0112; G01N 21/8806

(57) ABSTRACT

Provided is a sample manufacturing method that includes: a step of forming a hanging drop consisting of a liquid drop of a medium solution in a hanging state while causing at least one cell aggregate to be encapsulated in the liquid drop of the medium solution, the medium solution becoming substantially transparent upon gelling or solidifying; and a step of causing the hanging drop to gel or solidify by causing a promoting factor that promotes gelling or solidification of the medium solution to act on the hanging drop.

### CROSS-REFERENCE TO RELATED APPLICATIONS

This application is based on Japanese Patent Application No. 2017-120214, the contents of which are incorporated herein by reference.

### TECHNICAL FIELD

The present invention relates to a sample manufacturing method, a sample manufacturing kit, an observation method, and an observation device.

### BACKGROUND ART

In recent years, methods of obtaining microscope image data of three-dimensionally cultured cells, such as cell aggregates, screening the obtained microscope image data using image analysis techniques, and evaluating medicinal effects have attracted attention. As a method of manufacturing a cell aggregate, for example, there is a known method in which cells are dispensed together with a culture medium as a liquid drop onto the inner surface of a petri dish lid, a hanging drop is formed by inverting the liquid drop, and cell aggregation is induced inside the hanging drop with the help of the gravity component in a direction that extends along a curved surface of the hanging drop (for example, refer to PTL 1). However, since hanging drops are not precisely arranged in an array in this method, it is clear that this method is not suitable for automating the manufacture of cell aggregates.

Improving upon this issue in PTL 1, there is a known multiwell plate structure that can form hanging drops that are suitable for automation (for example, refer to PTL 2). The multiwell plate disclosed in PTL 2 is formed by arranging, in an array, sets that each consist of a recessed part that receives a liquid ejected from a dispenser, a hanging-drop forming section in which a hanging drop is formed and held, and a conduit that leads to the recessed part and the hanging-drop forming section. With the multiwell plate disclosed in PTL 2, there is no need to invert the liquid drops like in the method disclosed in PTL 1, hanging drops can be formed by simply dispensing cells, a culture medium, and so forth from above the multiwell plate in accordance with the array arrangement format, and consequently it is easy to automate the manufacture of cell aggregates in hanging drops. However, similarly to PTL 1, in PTL 2 there is no mention of a high-resolution observation method using a microscope.

There is a known technology that further develops the technology disclosed in PTL 2 and enables high-resolution observation and imaging to be easily performed using a microscope (for example, refer to PTL 3). In the technology disclosed in PTL 3, a manufactured cell aggregate inside a hanging drop is dropped into a well of a multiwell plate having a flat bottom surface together with the hanging drop and is observed and imaged using an inverted microscope via the bottom surface of the well. The well is designed such that a lateral cross section thereof gradually becomes narrower in a downward direction and so as to be shaped such that the surface area of the bottom surface of the well is slightly larger than the cell aggregate, and as a result the XY position of the cell aggregate that has been dropped onto the bottom surface of the well (position in directions that intersect vertical direction) can be roughly fixed. Thus, the cell aggregate is readily aligned with an observation optical axis.

### CITATION LIST

### Patent Literature

{PTL 1} German Patent No. 10362002
{PTL 2} The Publication of Japanese Patent No. 5490803
{PTL 3} PCT International Publication No. 2017/001880

### SUMMARY OF INVENTION

The present invention provides the following solutions.

A first aspect of the present invention provides a sample manufacturing method that includes: a step of forming a hanging drop consisting of a liquid drop of a medium solution in a hanging state while causing at least one cell aggregate to be encapsulated in the liquid drop of the medium solution, the medium solution becoming substantially transparent upon gelling or solidifying; and a step of causing the hanging drop to gel or solidify by causing a promoting factor that promotes gelling or solidification of the medium solution to act on the hanging drop.

A second aspect of the present invention provides a sample manufacturing method that includes: a step of forming a hanging drop consisting of a liquid drop of a culture medium in a hanging state while causing at least one cell to be encapsulated in the liquid drop of the culture medium; a step of culturing the cell inside the hanging drop until a desired cell aggregate is formed; a step of adding to the hanging drop a medium solution that becomes substantially transparent upon gelling or solidifying; and a step of causing the hanging drop to gel or solidify by causing a promoting factor that promotes gelling or solidification of the medium solution to act on the hanging drop.

A third aspect of the present invention provides a sample manufacturing method that includes: a step of forming a hanging drop consisting of a liquid drop of a culture medium and a medium solution in a hanging state while causing at least one cell to be encapsulated in the liquid drop of the culture medium and the medium solution, the medium solution becoming substantially transparent upon gelling or solidifying; a step of culturing the cell inside the hanging drop until a desired cell aggregate is formed; and a step of causing the hanging drop to gel or solidify by causing a promoting factor that promotes gelling or solidification of the medium solution to act on the hanging drop.

A fourth aspect of the present invention provides a sample manufacturing kit that includes: a hanging-drop forming implement having a recessed part into which a solution is injected, a hanging-drop forming section that holds a liquid drop of the solution injected in the recessed part in a hanging state while causing a cell aggregate to be encapsulated inside the liquid drop, and a conduit that connects the recessed part and the hanging-drop forming section to each other; a medium solution that is injected into the recessed part together with a cell and becomes substantially transparent upon gelling or solidifying; and a promoting solution that promotes gelling or solidification of the medium solution.

A fifth aspect of the present invention provides an observation method in which a hanging drop consisting of a liquid drop in a hanging state in which a cell aggregate is encapsulated is held, and observation light from the cell aggregate inside the hanging drop is detected.

A sixth aspect of the present invention provides an observation device that includes: a hanging-drop forming implement that forms a hanging drop consisting of a liquid drop in a hanging state in which a cell aggregate is encapsulated; a detection optical system that detects observation light emitted from the cell aggregate encapsulated inside the hanging drop formed by the hanging-drop forming implement; and a driving device that changes a relative position of the hanging drop held by the hanging-drop forming implement and a detection position of the detection optical system.

### BRIEF DESCRIPTION OF DRAWINGS

FIG. 1 is a flowchart for explaining a sample manufacturing method according to a first embodiment of the present invention.

FIG. 2 is a vertical sectional view of a hanging-drop forming implement used in the sample manufacturing method according to the first embodiment of the present invention.

FIG. 3 is a vertical sectional view of a hanging-drop forming implement used in a sample manufacturing method according to a modification of the first embodiment of the present invention.

FIG. 4 is a vertical sectional view illustrating an example in which a hanging drop is caused to gel or solidify by being irradiated with light, as a modification of the first embodiment of the present invention.

FIG. 5 is a flowchart for explaining a sample manufacturing method according to a first modification of the first embodiment of the present invention.

FIG. 6 is a vertical sectional view of a hanging-drop forming implement and a hanging drop for explaining the sample manufacturing method according to the first modification of the first embodiment of the present invention.

FIG. 7 is a vertical sectional view of a hanging-drop forming implement and a hanging drop for explaining a sample manufacturing method according to a second modification of the first embodiment of the present invention.

FIG. 8 is a vertical sectional view of a hanging-drop forming implement and a hanging drop for explaining a sample manufacturing method according to a third modification of the first embodiment of the present invention.

FIG. 9 is a flowchart for explaining the sample manufacturing method according to the third modification of the first embodiment of the present invention.

FIG. 10 is a diagram illustrating, in outline, the configuration of an observation device according to a second embodiment of the present invention.

FIG. 11 is a diagram illustrating, in outline, the configuration of an observation device according to a third embodiment of the present invention.

FIG. 12 is a plan view in which an adjustable diaphragm in FIG. 11 is viewed in a direction along an illumination optical axis.

FIG. 13 is a diagram illustrating, in outline, the configuration of an observation device according to a fourth embodiment of the present invention.

FIG. 14 is a diagram illustrating, in outline, the configuration of an observation device according to a first modification of the fourth embodiment of the present invention.

FIG. 15 is a diagram illustrating, in outline, the configuration of an observation device according to a third modification of the fourth embodiment of the present invention.

### DESCRIPTION OF EMBODIMENTS

### First Embodiment

Hereafter, a sample manufacturing method according to a first embodiment of the present invention will be described while referring to the drawings.

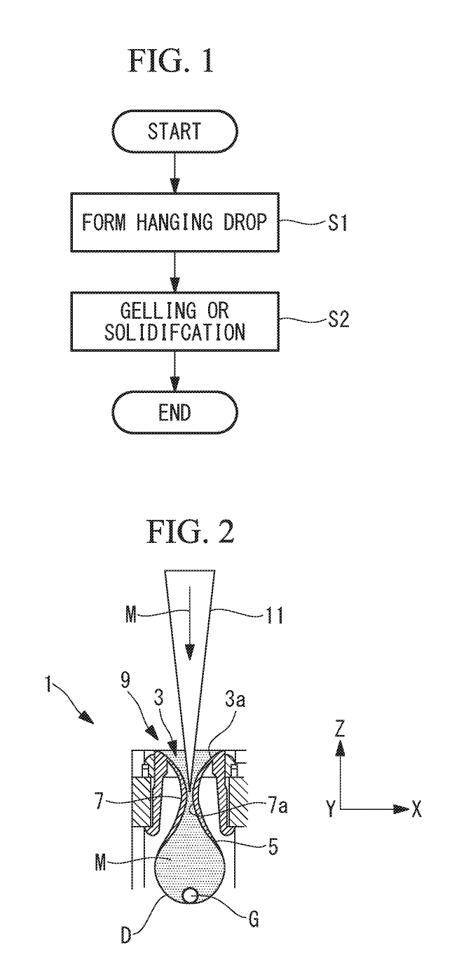

As illustrated in the flowchart in FIG. 1 and in FIG. 2, the sample manufacturing method according to this embodiment includes step S1 of forming a hanging drop D consisting of a liquid drop of a medium solution M in a hanging state while causing at least one cell aggregate G to be encapsulated in the liquid drop of the medium solution M, the medium solution M becoming substantially transparent upon gelling or solidifying, and step S2 of causing the hanging drop D to gel or solidify.

In this sample manufacturing method, the hanging drop D is formed using a hanging-drop forming implement 1 as illustrated in FIG. 2, for example.

The hanging-drop forming implement 1 includes a recessed part 3 into which a solution is injected, a hanging-drop forming section 5 that holds a liquid drop of the solution injected into the recessed part 3 in a hanging state while causing a cell aggregate G to be encapsulated inside the liquid drop, and a thin conduit 7 that connects the recessed part 3 and the hanging-drop forming section 5 to each other.

The hanging drop forming implement 1 may be composed of one set of the recessed part 3, the hanging-drop forming section 5, and the conduit 7, or may be a multiwell plate formed by arranging such sets in an array. FIG. 2 illustrates one set of the recessed part 3, the hanging-drop forming section 5, and the conduit 7 of the hanging-drop forming implement 1, which is composed of a multiwell plate having an array structure. Hereafter, the set of the recessed part 3, the hanging-drop forming section 5, and the conduit 7 will be referred to as a hanging-drop forming unit 9.

The hanging-drop forming unit 9 is formed of the recessed part 3, the conduit 7, and the hanging-drop forming section 5, which are arranged in this order from the top in the vertical direction. Hereafter, the vertical direction will be referred to as a Z direction, and directions that intersect the Z direction and are perpendicular to each other will be referred to as an X direction and a Y direction.

The recessed part 3 has an opening 3a that opens vertically upwards and has a substantially conical shape that extends from the opening 3a to the conduit 7 while becoming narrower in a tapering shape vertically downward.

The conduit 7 has a through hole 7a that penetrates through the conduit 7 in the vertical direction.

The hanging-drop forming section 5 has a substantially conical shape that gradually becomes wider in a radial direction toward the outside as the hanging-drop forming section 5 extends vertically downward from the conduit 7.

For example, an agarose solution is used as the medium solution M. For example, an agarose solution has a gelling property such that the agarose solution gels when the temperature falls to around 32-45° C. and is transparent upon gelling. The medium solution M has a specific gravity of 1, which is lower than the specific gravity of the cell aggregate G.

In step S1 of forming a hanging drop D, the cell aggregate G is dispensed together with the medium solution M into the recessed part 3 of the hanging-drop forming implement 1.

In step S2 of causing the hanging drop D to gel or solidify, a temperature is made to act on the hanging drop D as a promoting factor.

The operation of the thus-configured sample manufacturing method will be described next.

In order to manufacture a sample using the sample manufacturing method according to this embodiment, first, at least one cell aggregate G, which was manufactured in advance, is dispensed together with the medium solution M, which is composed of an agarose solution, into the recessed part 3 of the hanging-drop forming implement 1 using a dispenser 11.

The medium solution M and the cell aggregate G dispensed into the recessed part 3 move under gravity into the hanging-drop forming section 5 via the through hole 7a of the conduit 7. Then, a hanging drop D that consists of a liquid drop of the medium solution M in a hanging state in which the cell aggregate G is encapsulated is formed (step S1).

Since the medium solution M constituting the hanging drop D has a lower specific gravity than the cell aggregate G, the cell aggregate G moves under gravity along the boundary of the hanging drop D and settles in the vicinity of the lowest point in the hanging drop D. Therefore, by deciding upon the quantity of medium solution M to be dispensed into the recessed part 3, not only can the position of the cell aggregate G in the X and Y directions be fixed but the position of the cell aggregate G in the Z direction can also be fixed. In addition, as a result of the hanging-drop forming implement 1 being used, there is no need to invert the liquid drop of the medium solution M in order to form the hanging drop D.

Next, the temperature of the hanging drop D held by the hanging-drop forming implement 1 is lowered in order to cause the hanging drop D to gel (step S2). Thus, a sample in which the position of the cell aggregate G has been fixed inside a substantially transparent hanging drop D is manufactured.

The hanging drop D may be allowed to gel naturally by setting the room temperature to lower than the gelling temperature of the hanging drop D and raising the temperature of the medium solution M to be higher than the gelling temperature when dispensing the medium solution M.

As described above, with the sample manufacturing method according to this embodiment, a sample in which the position of the cell aggregate G is fixed inside the substantially transparent hanging drop D can be manufactured by forming the hanging drop D by causing the cell aggregate G to be encapsulated in a liquid drop of the medium solution M and causing the hanging drop D to gel while the hanging drop D is held.

Then, the cell aggregate G can be observed with high resolution by detecting, outside the hanging drop D, light emitted from the cell aggregate G inside the hanging drop D. In addition, since a simple task of merely forming the hanging drop D by causing the cell aggregate G to be encapsulated in a liquid drop of the medium solution M and causing the promoting factor to act on the hanging drop D is performed, the manufacture of the sample can be automated. Consequently, a sample that allows the cell aggregate G to undergo high-resolution observation and imaging using a microscope can be easily manufactured, and the manufacture of the sample can be easily automated.

Furthermore, in the case where a multiwell plate in which the hanging-drop forming units 9 are arranged in an array is adopted as the hanging-drop forming implement 1, automatic dispensing is easy, and a large number of cell aggregates G that are to be screened can be imaged with high throughput.

In this case, a period of time is required until the position of the cell aggregate G finally settles inside the hanging drop D when causing the hanging drop D to gel or solidify. Accordingly, the method may further include, prior to causing the hanging drop D to gel or solidify, a step of adding to the medium solution M an inhibiting solution (not illustrated) that retards the gelling or solidification of the medium solution M by inhibiting promotion of gelling or solidification of the medium solution M.

By adding the inhibiting solution, the hanging drop D can be made to take a longer time to gel or solidify. Therefore, the hanging drop D can be caused to gel or solidify in a state where the cell aggregate G has become located in the vicinity of the lowermost point in the hanging drop D due to gravity and the position of the cell aggregate G in the Z direction and the X and Y directions inside the hanging drop D can be substantially fixed.

In the case where an agarose solution is used as the medium solution M, a solution in which condensed phosphate has been dissolved can be used as the inhibiting solution, for example.

In addition, although the hanging-drop forming implement 1 is described as an illustrative example in this embodiment, it is sufficient that the hanging-drop forming implement be able to fix the cell aggregate G inside the hanging drop D to enable observation and imaging and preferably be able to fix the cell aggregate G at a specific spatial position inside the hanging drop D, and the hanging-drop forming implement is not limited to the described configuration.

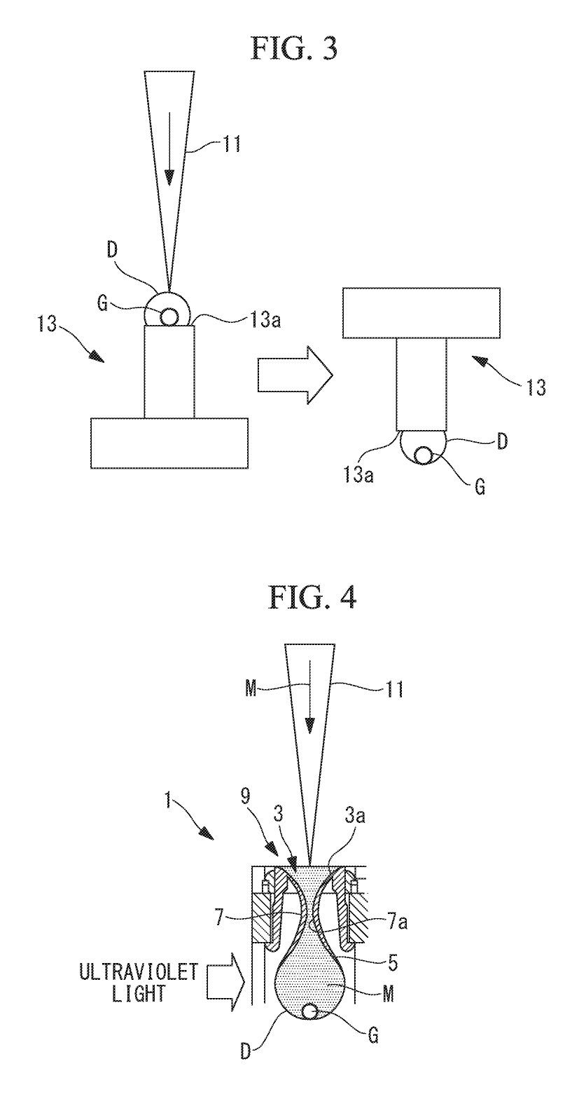

For example, as illustrated in FIG. 3, a rod 13 may be used as the hanging-drop forming implement. In this case, the hanging drop D may be formed by dispensing the medium solution M onto a rod end surface 13a using the dispenser 11 and then inverting the rod 13. With this configuration, there is an advantage that the structure of the hanging-drop forming implement can be simplified and formed at low cost.

In this modification, it is preferable that the rod end surface 13a be subjected to a water-repellent treatment such that a large hanging drop D can be formed. The rod 13 may be a multiwell plate that is formed by arranging a plurality of the rod end surfaces 13a in an array.

Furthermore, in this embodiment, for example, an ultraviolet-light-curable liquid resin may be employed as the medium solution M and light may serve as the promoting factor. In this case, as illustrated in FIG. 4, the hanging drop D may be formed of a liquid drop of the medium solution M composed of an ultraviolet-light-curable liquid resin and the hanging drop D may be solidified by irradiating the hanging drop D with ultraviolet radiation (light).

With this configuration, a simple task of merely irradiating the medium solution M with specific light is performed, and therefore the timing at which the hanging drop D is solidified can be freely set. Furthermore, there is an advantage in that a plurality of the hanging drops D can be solidified all at once by being irradiated with ultraviolet radiation, and screening can be performed with high throughput. In addition, there is also an advantage that the hanging drop D can be completely solidified, and the solidified hanging drop D can be easily stored.

Furthermore, in this embodiment, for example, a sodium alginate solution may be used as the medium solution M and the hanging drop D may be caused to gel or solidify through a chemical reaction using a calcium ion as the promoting factor.

This embodiment can be modified in the following ways.

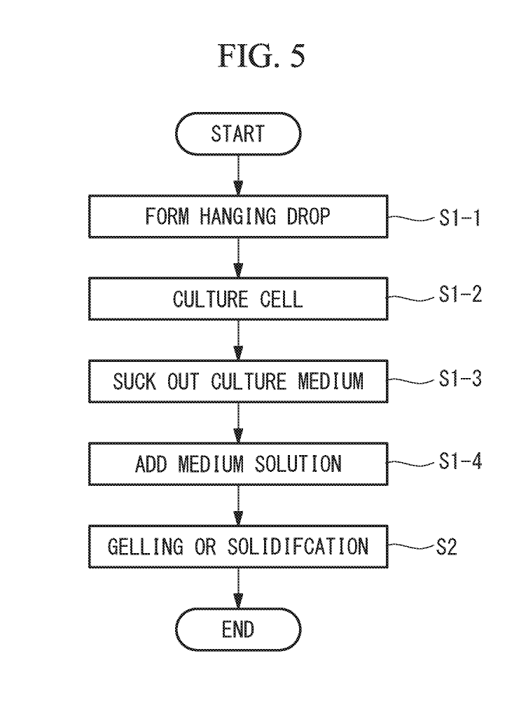

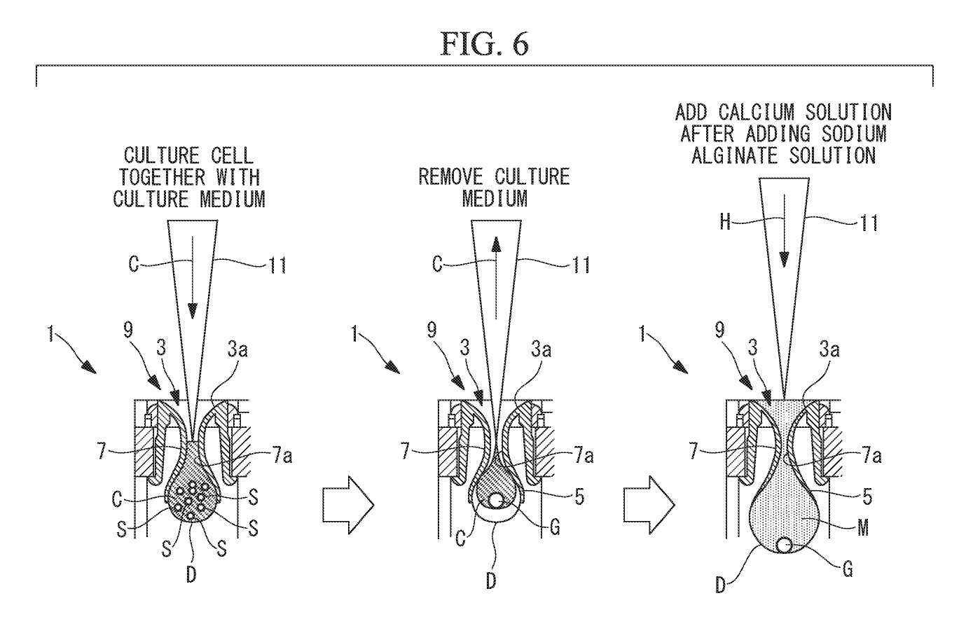

As a first modification, for example, the cell aggregate G may be manufactured by culturing a cell inside the hanging drop D. In other words, as illustrated in the flowchart in FIG. 5 and in FIG. 6, a sample manufacturing method according to this modification may include a step S1-1 of forming a hanging drop D consisting of a liquid drop of a culture medium C in a hanging state while causing at least one cell S to be encapsulated inside the liquid drop of the culture medium C, a step S1-2 of culturing the cell S inside the hanging drop D until a desired cell aggregate G is formed, a step S1-4 of adding to the hanging drop D a medium solution M that becomes substantially transparent upon gelling or solidifying, and the step S2 of causing the hanging drop D to gel or solidify. The sample manufacturing method according to this modification may further include a step S1-3 of sucking the culture medium C from the hanging drop D after culturing the cell S and prior to adding the medium solution M to the hanging drop D.

In this case, in step S1-1, at least one cell S may be dispensed together with the culture medium C into the recessed part 3 of the hanging-drop forming implement 1 using the dispenser 11.

In step S1-2, the hanging drop D may be maintained in a state of being held by the hanging-drop forming implement 1 until the cell S has cultured. The culture medium may be switched, as appropriate, in this step.

In step S1-3, some of the culture medium C may be removed by sucking the culture medium C out through the recessed part 3 using the dispenser 11 while leaving an amount of the culture medium C that allows the hanging drop D to be maintained.

In step S1-4, for example, an alginic-acid-based solution may be used as the medium solution M, and a sodium alginate solution may be added to the hanging drop D using the dispenser 11. The sodium alginate solution becomes transparent upon gelling.

In step S2, a promoting solution H may be caused to act on the hanging drop D as the promoting factor. For example, a calcium solution in which a calcium ion (promoting factor) has been dissolved may be used as the promoting solution H, and the hanging drop D may be caused to gel by additionally adding the calcium solution to the hanging drop D to which the sodium alginate solution serving as the medium solution M has been added.

According to this modification, the cell aggregate G is formed by culturing the cell S inside the hanging drop D formed of a liquid drop of the culture medium C, and consequently there is no need to move the cell aggregate G, throughput can be improved, and screening can be performed at low cost.

In addition, fluorescence is generated in the culture medium C by illumination light, and background light is increased in the case where fluorescence observation is performed, and therefore fluorescence observation is not preferred. Furthermore, there is a possibility of the culture medium C containing a component that will affect gelling or solidification of the hanging drop D. Therefore, gelling or solidification of the hanging drop D can be made easier by removing some of the culture medium C from the hanging drop D by sucking the culture medium C.

Although an alginic-acid-based solution is used as the medium solution M in this modification, the modification is not limited to this solution. For example, an epoxy-based liquid resin may be used as the medium solution M, and a polyamine solution in which polyamines have been dissolved may be used as the promoting solution H. In addition, the modification is not limited to causing the hanging drop D to gel or solidify by mixing two liquids, and the hanging drop D may instead be caused to gel or solidify by mixing a larger number liquids, for example, three or more.

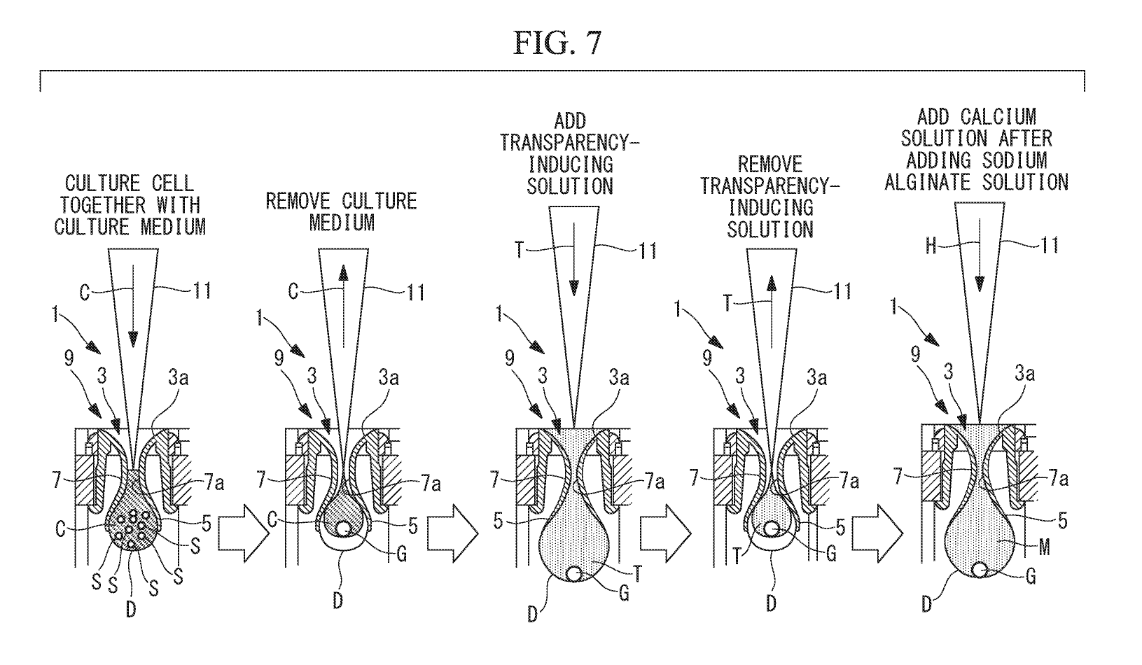

As a second modification, for example, as illustrated in FIG. 7, the method may include a step of adding a transparency-inducing solution T, which turns the cell aggregate G transparent, to the hanging drop D prior to causing the hanging drop D to gel or solidify by making the promoting factor act on the hanging drop D.

For example, in the case where a large cell aggregate G having a diameter exceeding 300 μm is to be observed, the illumination light (excitation light) used for observation may not be able to reach the inside of the cell aggregate G, and it may not be possible to observe the internal structure of the cell aggregate G. According to this modification, the illumination light used for observation can easily reach the inside of the cell aggregate G even in the case of a large cell aggregate G. Thus, the internal structure of the cell aggregate G can be easily observed regardless of the size of the cell aggregate G.

In this modification, as illustrated in FIG. 7, the method may further include a step of removing, by suction, at least some of the transparency-inducing solution T from the hanging drop D once the cell aggregate G has turned transparent. Thus, the hanging drop D can be easily caused to gel or solidify even in the case where the transparency-inducing solution T contains a component that affects gelling or solidification of the hanging drop D.

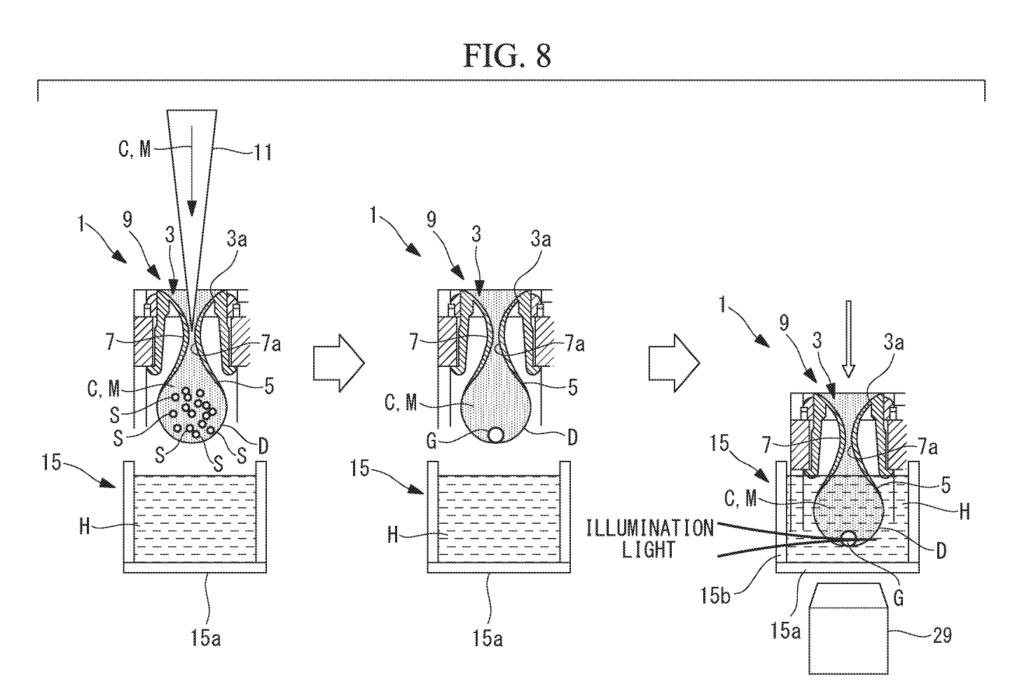

As a third modification, as illustrated in FIG. 8, the hanging drop D may be caused to gel or solidify by causing the hanging drop D to be immersed in (contacted by) the promoting solution H.

In this case, for example, a calcium solution may be used as the promoting solution H, a medium container 15 such as a cuvette having a bottom part (transparent part, observation-light-transmitting transparent part) 15a and a side wall part (transparent part, illumination-light-transmitting transparent part) 15b through which light can pass may be used, and the promoting solution H may be stored in the medium container 15 as follows.

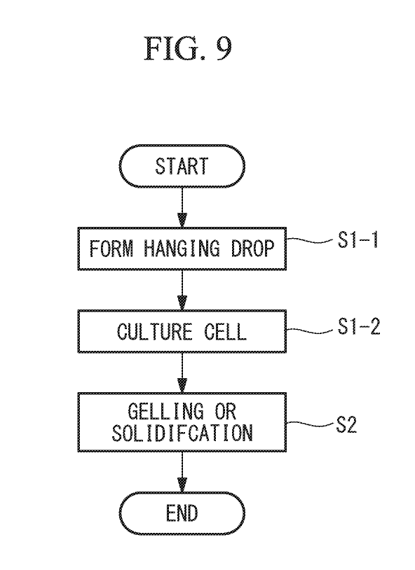

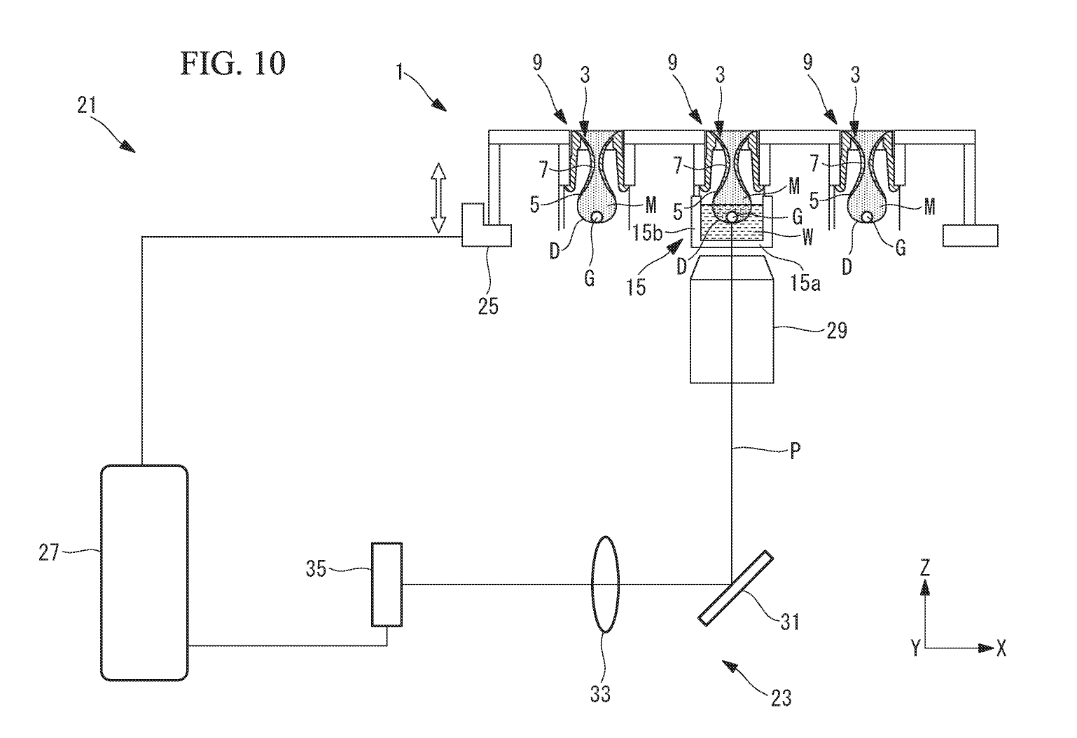

For example, as illustrated in the flowchart in FIG. 9, the hanging drop D may be formed by dispensing a sodium alginate solution, which serves as the medium solution M, and at least one cell S together with the culture medium C into the recessed part 3 of the hanging-drop forming implement 1 (step S1-1). The cell S is then cultured inside the hanging drop D until a desired cell aggregate G is formed (step S1-2).

Once the desired cell aggregate G is formed, the hanging drop D is caused to gel or solidify by slowly immersing the hanging drop D in the promoting solution H stored inside the medium container 15 (step S2). Light emitted vertically downward from the cell aggregate G can be observed via the bottom part 15a of the medium container 15 using a microscope. Reference symbol 29 in FIG. 8 denotes an objective lens.

According to this modification, there is no need to dispense the promoting solution H into the hanging drop D, and the hanging drop D can be caused to gel or solidify by simply lowering the hanging drop D so as to be immersed in the promoting solution H. Therefore, in the case where a multiwell plate having an array structure is used as the hanging-drop forming implement 1, a plurality of hanging drops D can be caused to gel or solidify by being immersed in the promoting solution H all at once.

Furthermore, the cell aggregates G can be observed using a light-sheet microscope with the hanging drops D remaining immersed in the medium container 15, and this setup is suitable for large volume screening.

In each of the above-described modifications, a sample manufacturing kit (not illustrated) that includes the hanging-drop forming implement 1, the medium solution M, and the promoting solution H can be formed.

With the thus-configured sample manufacturing kit, a sample in which a cell aggregate G can be observed and imaged at high resolution using a microscope can be easily manufactured, and the manufacture of the sample can be easily automated.

### Second Embodiment

Next, an observation device and an observation method according to a second embodiment of the present invention will be described.

Hereafter, parts of the configuration that are common to the sample manufacturing method according to the first embodiment are denoted by the same reference symbols, and a description thereof is omitted.

As illustrated in FIG. 10, an observation device 21 according to this embodiment is configured as an inverted microscope. The observation device 21 includes the hanging-drop forming implement 1, the medium container 15, a detection optical system 23 that detects observation light emitted from a cell aggregate G encapsulated inside a substantially transparent gelled or solidified hanging drop D formed by the hanging-drop forming implement 1, a driving device 25 that changes the relative position of the hanging drop D held by the hanging-drop forming implement 1 and a detection position of the detection optical system 23, and a controller (control unit) 27 that controls the detection optical system 23, the driving device 25, and so forth.

The hanging-drop forming implement 1 is formed of a multiwell plate in which the hanging-drop forming units 9, which are each constituted by the recessed part 3, the hanging-drop forming section 5, and the conduit 7, are arranged in an array. In the example illustrated in FIG. 10, three hanging-drop forming units 9 are arranged in the X direction, and four hanging-drop forming units 9 are arranged in the Y direction.

The driving device 25 is a motor-driven stage and supports the hanging-drop forming implement 1 so that the hanging-drop forming implement 1 can be moved in the X, Y, and Z directions.

The medium container 15 is arranged along a detection optical axis P, which extends in the Z direction, of the detection optical system 23. A liquid immersion medium W, which has the same refractive index as the solution constituting the hanging drops D, is stored in the medium container 15.

In this embodiment, the medium container 15 is of such a size that hanging drops D held by four hanging-drop forming units 9 arranged in the Y direction can be selectively arranged on the detection optical axis P by moving the hanging-drop forming implement 1 in the Y direction. Thus, four hanging drops D can be selectively arranged on the detection optical axis P by simply moving the hanging-drop forming implement 1 in the Y direction using the driving device 25 without moving the hanging-drop forming implement 1 in the Z direction.

The liquid immersion medium W includes a luminescent substrate. A luminescence gene is introduced into the part of the cell aggregate G used in this embodiment that is to be observed, and bioluminescence is produced when the hanging drop D is immersed in the liquid immersion medium W.

The detection optical system 23 includes an objective lens 29 that is arranged on the detection optical axis P so as to face the bottom part 15a of the medium container 15, a reflecting mirror 31 that reflects light collected by the objective lens 29, an image-forming lens 33 that forms an image of the light reflected by the reflecting mirror 31, and a camera 35 that captures the image of the light formed by the image-forming lens 33.

For example, the controller 27 includes a central processing unit (CPU), a main storage unit such as a read only memory (ROM) or a random access memory (RAM), an auxiliary storage unit such as a hard disk drive (HDD), an input unit with which a user inputs instructions, an output unit that outputs data, and an external interface that exchanges various types of data with an external device (none of which are illustrated). Various programs are stored in the auxiliary storage unit. The CPU reads a program from the auxiliary storage unit into the main storage unit such as a RAM and executes the program in order to realize various processing operations.

Specifically, the controller 27 causes the hanging-drop forming implement 1 to move by driving the driving device 25 and arranges the cell aggregate G of the hanging drop D that is to be observed on the detection optical axis P by executing a program. In addition, the controller 27 generates an image by controlling the camera 35.

Next, in the observation method according to this embodiment, the hanging drop D that consists of a liquid drop of the medium solution M in a hanging state, in which the cell aggregate G has been encapsulated, is held, the hanging drop D that is to be observed is immersed inside the liquid immersion medium W in the medium container 15, and observation light from the cell aggregate G inside the hanging drop D is detected.

The operation of the thus-configured observation device 21 and observation method will be described.

When a cell aggregate G is to be observed using the observation device 21 and observation method according to this embodiment, first, a gelled or solidified substantially transparent hanging drop D consisting of a liquid drop of the medium solution M in a hanging state, in which the cell aggregate G is encapsulated, is held by the hanging-drop forming implement 1 in each hanging-drop forming unit 9.

Then, the controller 27 moves the hanging-drop forming implement 1 by driving the driving device 25 so as to immerse the hanging drop D, in which the cell aggregate G that is to be observed is encapsulated, in the liquid immersion medium W in the medium container 15, and arranges the hanging drop D on the detection optical axis P.

A luminescence gene is introduced into the part of the cell aggregate G that is to be observed, and a luminescent substrate is included in the liquid immersion medium W, and therefore the cell aggregate G generates bioluminescence when the hanging drop D is immersed in the liquid immersion medium W. Luminescence radiated vertically downward out of the luminescence generated in the part of the cell aggregate G that is to be observed is collected by the objective lens 29 after passing through the liquid immersion medium W and the transparent bottom part 15a of the medium container 15. The luminescence collected by the objective lens 29 is reflected by the reflecting mirror 31 and is formed into an image on an image-capturing plane of the camera 35 by the image-forming lens 33. Thus, an observation image of the cell aggregate G is obtained in the camera 35.

Tomographic images at respective observation positions can be acquired by changing the observation position of the cell aggregate G by moving the hanging drop D in the X, Y, and Z directions inside the medium container 15 by driving the driving device 25 using the controller 27. In addition, a cell aggregate G encapsulated in another hanging drop D can be observed by changing the hanging drop D that is arranged on the detection optical axis P by moving the hanging-drop forming implement 1 in the X, Y, and Z directions.

As described above, with the observation device 21 and observation method according to this embodiment, the cell aggregate G can be observed by detecting luminescence from the cell aggregate G inside the hanging drop D using the detection optical system 23. Therefore, a plurality of samples can be observed in a short period of time and at low cost without the use of wells into which hanging drops D are dropped.

Although the medium container 15 having the transparent bottom part 15a and side wall part 15b is exemplified as the medium container in this embodiment, a medium container that has an observation-light-transmitting transparent part through which light on the detection optical axis P can pass in at least the bottom part of the medium container may instead be used.

### Third Embodiment

Next, an observation device and an observation method according to a third embodiment of the present invention will be described.

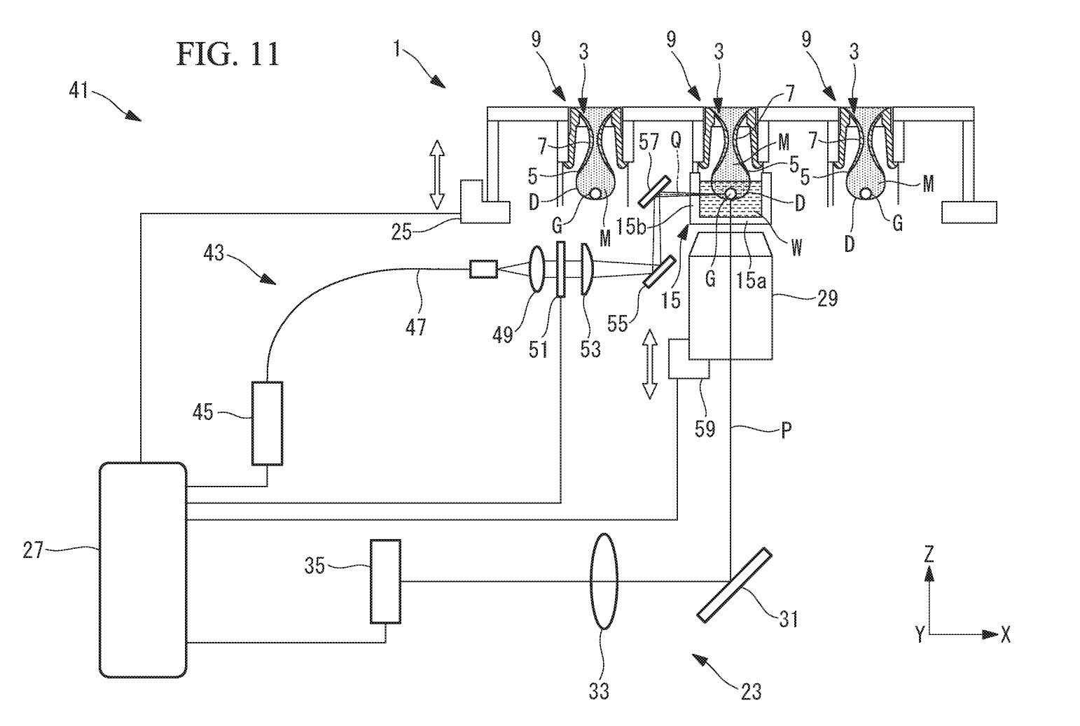

As illustrated in FIG. 11, an observation device 41 differs from the second embodiment in that an inverted light-sheet microscope is formed.

Hereafter, parts of the configuration that are common to the sample manufacturing method according to the first embodiment and the observation device 21 and observation method according to the second embodiment are denoted by the same reference symbols, and a description thereof is omitted.

The observation device 41 includes the hanging-drop forming implement 1, the medium container 15, the driving device 25, an illumination optical system 43, the detection optical system 23, and the controller 27.

The illumination optical system 43 includes a laser light source 45 that generates laser light, an optical fiber 47 that guides the laser light emitted from the laser light source 45, a collimating lens 49 that converts the laser light emitted from the emission end of the optical fiber 47 into a parallel light beam, an adjustable diaphragm 51 that can change the light beam diameter of the laser light converted into parallel light beam by the collimating lens 49, a cylindrical lens 53 that collects the laser light that has passed through the adjustable diaphragm 51 in a planar shape along a plane that is perpendicular to the detection optical axis P, and two reflecting mirrors 55 and 57 that reflect the laser light collected by the cylindrical lens 53 and make the laser light incident on a hanging drop D after passing through the transparent side wall part 15b of the medium container 15 along an illumination optical axis Q, which is perpendicular to the detection optical axis P.

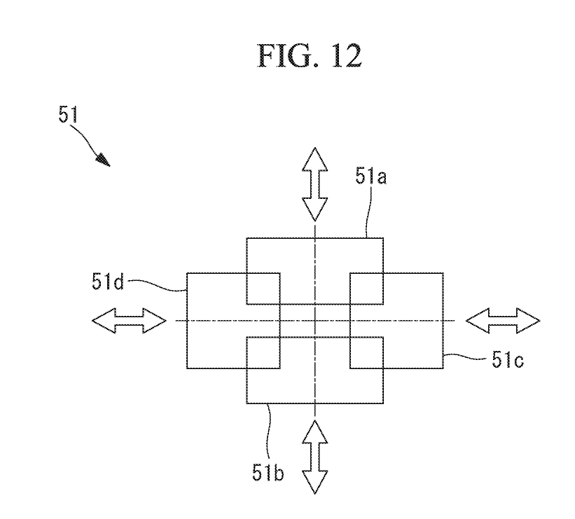

As illustrated in FIG. 12, the adjustable diaphragm 51 has four light-blocking blades 51a, 51b, 51c, and 51d. The light beam diameter of the laser light can be changed by moving the four light-blocking blades 51a, 51b, 51c, and 51d in directions that intersect the optical axis of the collimating lens 49. The thickness and width of the laser light collected in a planar shape by the cylindrical lens 53 can be changed by changing the light beam diameter of the laser light using the adjustable diaphragm 51.

The cylindrical lens 53 has power in one direction that is perpendicular to the illumination optical axis Q of the illumination optical system 43. The cylindrical lens 53 forms a focal point on the detection optical axis P of the detection optical system 23 by collecting the laser light composed of a substantially parallel light beam in a planar shape having a prescribed width dimension equal to the light beam diameter of the laser light.

The detection optical system 23 includes a sighting unit 59 that allows the objective lens 29 to be moved in directions along the detection optical axis P. The sighting unit 59 enables fine adjustment of the focal position of the objective lens 29 in directions along the detection optical axis P by finely moving the objective lens 29 in directions along the detection optical axis P.

In addition to controlling the laser light source 45 and the camera 35, controlling the driving device 25, and generating images by executing a program, the controller 27 also adjusts the light beam diameter of the laser light using the adjustable diaphragm 51 and finely adjusts the position of the objective lens 29 in a direction along the detection optical axis P of the detection optical system 23 using the sighting unit 59.

The operation of the thus-configured observation device 41 and observation method will be described.

When a cell aggregate G is to be observed using the observation device 41 and observation method according to this embodiment, first, the controller 27 causes a gelled or solidified substantially transparent hanging drop D, in which the cell aggregate G that is to be observed is encapsulated, to be immersed in the liquid immersion medium W inside the medium container 15 by moving the hanging-drop forming implement 1 by driving the driving device 25, arranges the cell aggregate G on the illumination optical axis Q and the detection optical axis P, and causes laser light to be generated from the laser light source 45.

The laser light emitted from the laser light source 45 is guided by the optical fiber 47 and converted into a parallel light beam by the collimating lens 49, and the light beam diameter is restricted by the adjustable diaphragm 51. Having passed through the adjustable diaphragm 51, the laser light is collected in a planar shape by the cylindrical lens 53, reflected by the reflecting mirrors 55 and 57, passes through the side wall part 15b of the medium container 15, and is enters the medium container 15.

The laser light that has entered the medium container 15 is incident on the cell aggregate G inside the hanging drop D from a direction perpendicular to the detection optical axis P after passing through the liquid immersion medium W. As a result of the planar laser light being incident on the cell aggregate G, a fluorescent substance inside the cell aggregate G is excited along the incidence plane of the laser light, and fluorescence (observation light) is generated.

Of the fluorescence generated in the cell aggregate G, fluorescence radiated in a direction along the detection optical axis P is collected by the objective lens 29 after passing through the medium solution M and the bottom part 15a of the medium container 15 from the hanging drop D. The fluorescence collected by the objective lens 29 is reflected by the reflecting mirror 31 and is formed into an image on an image-capturing plane of the camera 35 by the image-forming lens 33. Thus, a tomographic image perpendicular to the detection optical axis P of the cell aggregate G is obtained by the camera 35.

In this case, it is preferable that the cell aggregate G be arranged with a space between the cell aggregate G and the bottom part 15a of the medium container 15. If the cell aggregate G is in contact with the bottom part 15a of the medium container 15, the part of the cell aggregate G that is in contact with the bottom part 15a cannot be satisfactorily illuminated unless the refractive index of the bottom part 15a is equal to the refractive index of the liquid immersion medium W. By arranging the cell aggregate G so that there is a space between the cell aggregate G and the bottom part 15a of the medium container 15, the entire cell aggregate G can be satisfactorily observed regardless of the refractive index of the bottom part 15a of the medium container 15.

Tomographic images at respective observation positions can be acquired by changing the observation position of the cell aggregate G by moving the hanging drop D in the X, Y, and Z directions inside the medium container 15 by driving the driving device 25 using the controller 27. In addition, a cell aggregate G encapsulated in another hanging drop D can be observed by changing the hanging drop D that is arranged on the detection optical axis P by moving the hanging-drop forming implement 1 in the X, Y, and Z directions.

Here, the focal position of the cylindrical lens 53 and the optical axis of the objective lens 29 (detection optical axis P) are aligned with each other, and the focal plane of the objective lens 29 is aligned with the incidence plane of the laser light, and as a result fluorescence generated across a wide area of the focal plane of the objective lens 29 is collected all at once by the objective lens 29 and captured by the camera 35, and a sharp fluorescence image of the part of the cell aggregate G being observed can be obtained. In addition, since the laser light is not radiated outside the image-capturing plane of the camera 35, fading of the fluorescence can be suppressed, and an excellent three-dimensional image can be obtained.

Furthermore, when the cell aggregate G is moved in the Z direction in order to obtain XYZ stack images of the cell aggregate G, in the case where there is a difference between the refractive index of the medium solution M constituting the hanging drop D and the refractive index of the liquid immersion medium W, the incidence plane of the laser light and the focal plane of the objective lens 29 may become misaligned. In this case, the shift in the focal position can be eliminated by finely adjusting the position of the objective lens 29 in directions along the detection optical axis P by driving the sighting unit 59 using the controller 27.

In this embodiment, for example, the illumination optical system 43 may include a scanning member that scans the laser light in directions that intersect the illumination optical axis Q and may form laser light having a width in directions that intersect the illumination optical axis Q in accordance with the optical scanning.

### Fourth Embodiment

Next, an observation device and an observation method according to a fourth embodiment of the present invention will be described.

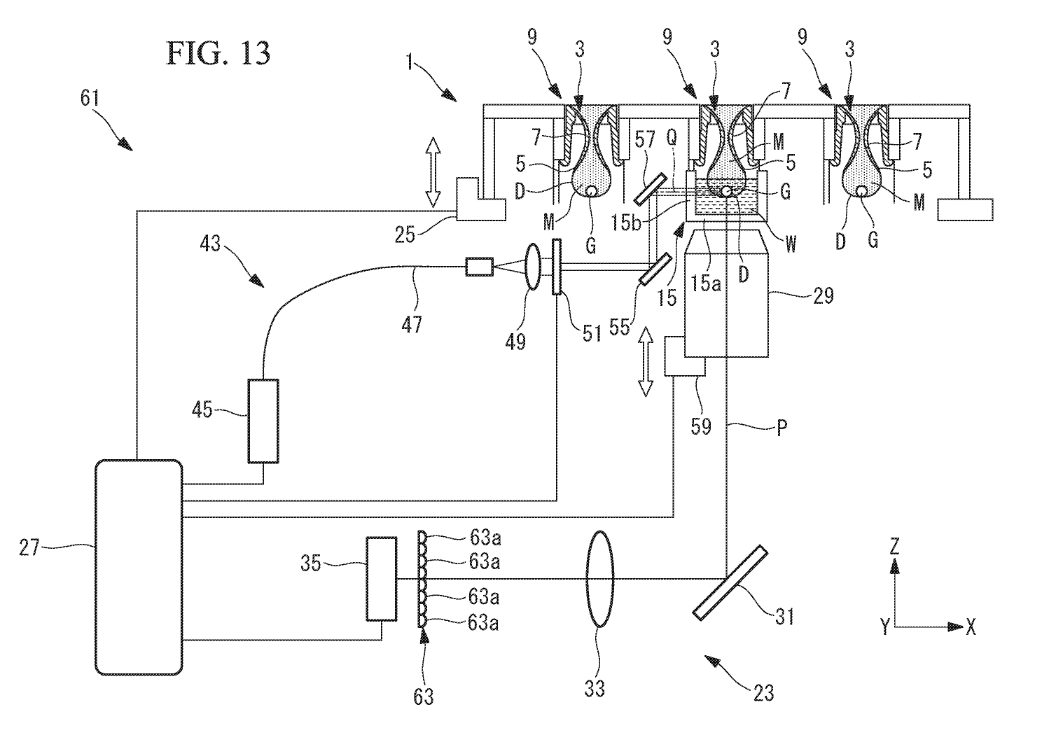

As illustrated in FIG. 13, an observation device 61 differs from the third embodiment in that an inverted light-field microscope is formed.

Hereafter, parts of the configuration that are common to the sample manufacturing method according to the first embodiment and the observation devices 21 and 41 and observation methods according to the second and third embodiments are denoted by the same reference symbols, and a description thereof is omitted.

The observation device 61 includes the hanging-drop forming implement 1, the medium container 15, the driving device 25, the illumination optical system 43, the detection optical system 23, and the controller 27.

The illumination optical system 43 includes the laser light source 45, the optical fiber 47, the collimating lens 49, the adjustable diaphragm 51, and the two reflecting mirrors 55 and 57.

The detection optical system 23 includes the objective lens 29, the reflecting mirror 31, the image-forming lens 33, a microlens array 63 composed of a plurality of microlenses 63a, and the camera 35. In addition, the objective lens 29 is equipped with the sighting unit 59.

The image-forming lens 33 is arranged to as to form an image of the fluorescence from the reflecting mirror 31 on the microlens array 63.

The microlens array 63 is arranged substantially at the focal position of the objective lens 29, fluorescence formed into an image by the image-forming lens 33 is collected by the plurality of microlenses 63a so that the image is projected onto the image-capturing plane of the camera 35.

The operation of the thus-configured observation device 61 and observation method will be described.

When a cell aggregate G is to be observed using the observation device 61 and observation method according to this embodiment, first, the controller 27 causes a gelled or solidified substantially transparent hanging drop D, in which the cell aggregate G that is to be observed is encapsulated, to be immersed in the liquid immersion medium W inside the medium container 15 by moving the hanging-drop forming implement 1 by driving the driving device 25, arranges the cell aggregate G on the illumination optical axis Q and the detection optical axis P, and causes laser light to be generated from the laser light source 45.

Laser light emitted from the laser light source 45 is guided by the optical fiber 47, converted into a parallel light beam by the collimating lens 49, and is emitted as a parallel light beam after being given a width in both the Y direction and the Z direction by the adjustable diaphragm 51, and the laser light is then reflected by the reflecting mirrors 55 and 57 and is made to enter the medium container 15 after passing through the side wall part 15b of the medium container 15.

The laser light that has entered the medium container 15 is incident on the cell aggregate G inside the hanging drop D from a direction perpendicular to the detection optical axis P of the detection optical system 23 after passing through the liquid immersion medium W. Fluorescence generated in the cell aggregate G by the incident light and radiated in a direction along the detection optical axis P is collected by the objective lens 29 after passing through the medium solution M and the bottom part 15a of the medium container 15 from the hanging drop D.

The fluorescence collected by the objective lens 29 is reflected by the reflecting mirror 31, is formed into an image on each microlens 63a of the microlens array 63 by the image-forming lens 33, and is projected onto the image-capturing plane of the camera 35. Thus, image capture data of the cell aggregate G is obtained by the camera 35, the image capture data is sent to the controller 27 and is subjected to recovery processing, and three-dimensional data is constructed.

As described above, with the observation device 61 and observation method according to this embodiment, a plurality of sets of image information of the cell aggregate G having different parallaxes can be obtained all at once.

The light-field microscope can acquire data at a prescribed depth in the Z direction in the cell aggregate G in one go, but image data may be acquired by moving the hanging-drop forming implement 1 in the Z direction using the driving device 25 if the depth at which data can be obtained is insufficient in terms of sample volume. In addition, in the case where a shift occurs in the focal point of the objective lens 29 as a result of the hanging-drop forming implement 1 being moved in the Z direction, this focal point shift may be adjusted using the sighting unit 59.

The second, third, and fourth embodiments described above can be modified in the following ways.

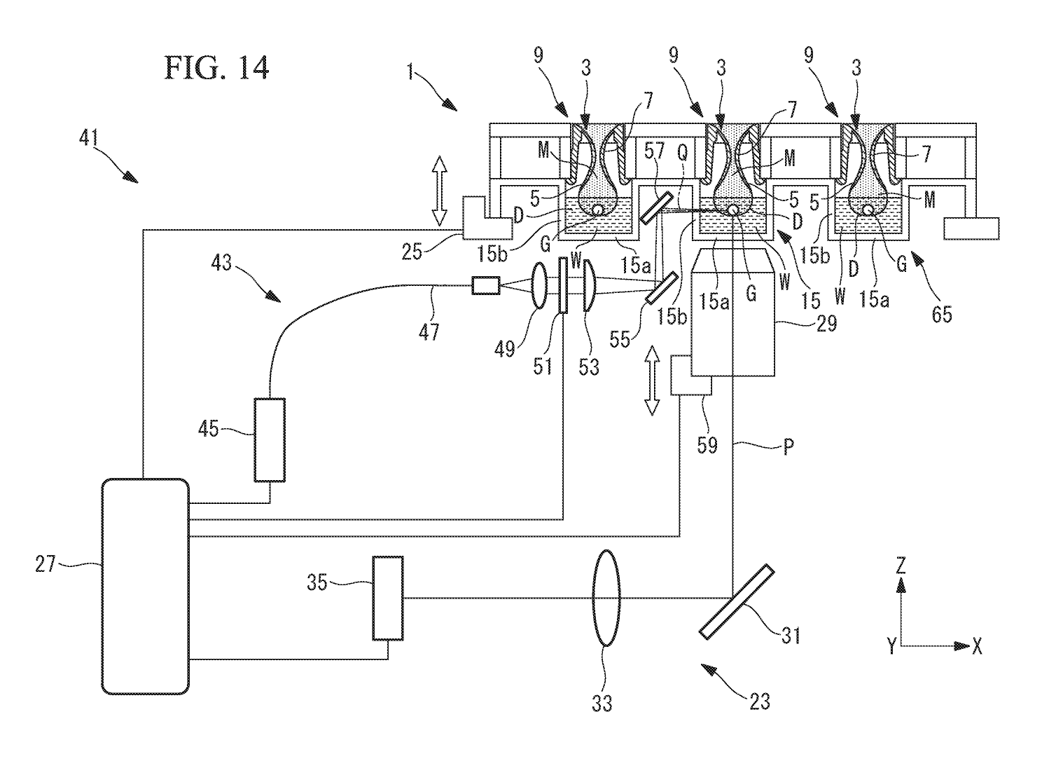

As a first modification, for example, as illustrated in FIG. 14, a medium container multiwell plate 65 in which a plurality of medium containers 15 are arranged so as to respectively correspond to the plurality of hanging-drop forming units 9 of the hanging-drop forming implement 1 may be used. FIG. 14 illustrates the observation device 41 as an example.

In the example illustrated in FIG. 14, in the medium container multiwell plate 65, three medium containers 15 are arranged in the X direction and four medium containers 15 are arranged in the Y direction so as to correspond to the arrangement of the hanging-drop forming units 9. The hanging-drop forming implement 1 is mounted on the medium container multiwell plate 65, and the hanging drops D held in the respective hanging-drop forming units 9 are immersed in the liquid immersion mediums W of the corresponding medium containers 15. The medium container multiwell plate 65 is supported so as to be capable of being moved by the driving device 25 in the X, Y, and Z directions together with the hanging-drop forming implement 1.

With this configuration, a desired cell aggregate G can be arranged on the detection optical axis P and observed by moving the medium container multiwell plate 65 in the X, Y, and Z directions together with the hanging-drop forming implement 1 using the driving device 25. In addition, since the hanging drops D are always immersed in the liquid immersion mediums W, screening can be performed while preventing the gelled or solidified hanging drops D from drying out.

In this modification, the liquid immersion medium W may include a culture medium. In this case, the medium solution M constituting the hanging drops D may be composed of a substance through which a culture component of the culture medium can pass. With this configuration, necessary gas replacement and component supply can be performed via the hanging drops D. Thus, time lapse observation can be performed while culturing a cell aggregate G.

As a second modification, the controller 27 may execute a program in order to drive the driving device 25 and move the hanging-drop forming implement 1 in order to sequentially align each cell aggregate G with the detection optical axis P of the detection optical system 23 by changing the relative position of the hanging drop D and the detection position of the detection optical system 23.

With this configuration, a plurality of cell aggregates G can be observed by sequentially aligning the cell aggregates G with the detection optical axis P of the detection optical system 23.

In addition, the controller 27 may execute sequential control for performing time lapse observation of photographing a cell aggregate G at prescribed time intervals on the basis of a program.

With this configuration, temporal changes in the cell aggregate G encapsulated in the hanging drop D can be observed.

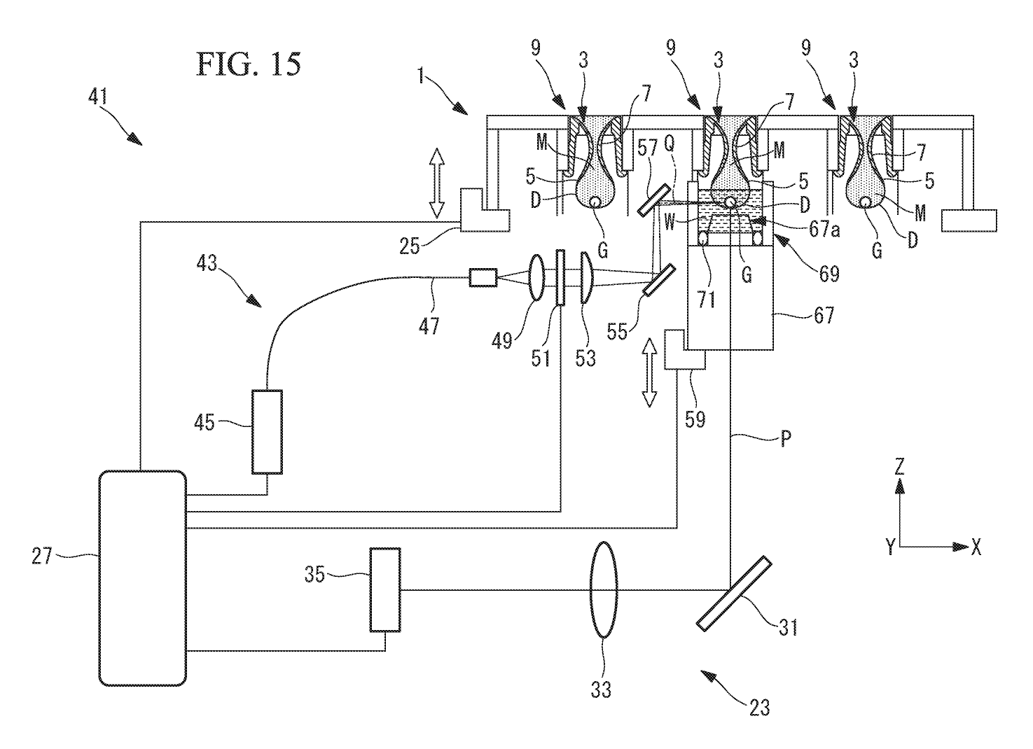

Furthermore, as a third modification, for example, as illustrated in FIG. 15, a liquid-immersion objective lens 67 may be used as the objective lens, the liquid-immersion objective lens 67 being arranged such that the optical axis thereof faces substantially vertically upward. In addition, the medium container 15 may be supported by the liquid-immersion objective lens 67. Specifically, a leading end 67a of the liquid-immersion objective lens 67 and a cylindrical member 69 that is attached to one end of the leading end 67a in the axial direction may form the medium container.

In this case, a shield member 71 such as an O ring may be arranged in a gap between the leading end 67a and the cylindrical member 69. In addition, the cylindrical member 69 may be formed of a material through which light can pass or may have a transparent part through which light on the illumination optical axis Q can pass (illumination-light-transmitting transparent part).

With this configuration, a medium container can be formed at low cost by using the leading end 67a of the liquid-immersion objective lens 67 as the bottom part of the medium container.

Embodiments of the present invention have been described in detail above while referring to the drawings, but the specific configuration of the present invention is not limited to these embodiments, and design changes and so forth that do not depart from the scope of the present invention are also included in the present invention. For example, the present invention is not limited to being applied as described in the above-described embodiments and modifications, and the present invention may be applied to an embodiment obtained by suitably combining any of the above-described embodiments and modifications thereof and is not particularly limited.

Furthermore, for example, although the medium container 15 has the transparent bottom part 15a and side wall part 15b in the above-described embodiments, the entire bottom portion and the entire side wall portion do not have to be transparent, and the medium container 15 may instead have an illumination-light-transmitting transparent part through which laser light from the illumination optical system 43 can pass and an observation-light-transmitting transparent part through which observation light from the cell aggregate G can pass.

As a result, the following aspects are derived from the above-described embodiments.

A first aspect of the present invention provides a sample manufacturing method that includes: a step of forming a hanging drop consisting of a liquid drop of a medium solution in a hanging state while causing at least one cell aggregate to be encapsulated in the liquid drop of the medium solution, the medium solution becoming substantially transparent upon gelling or solidifying; and a step of causing the hanging drop to gel or solidify by causing a promoting factor that promotes gelling or solidification of the medium solution to act on the hanging drop.

According to the first aspect of the present invention, a sample in which the position of a cell aggregate inside a substantially transparent hanging drop is fixed is manufactured by forming a hanging drop by encapsulating a cell aggregate in a liquid drop of a medium solution and gelling or solidifying the hanging drop by using a promoting factor.

Therefore, the cell aggregate can be observed at high resolution by detecting, outside the hanging drop, light emitted from the cell aggregate inside the hanging drop. In addition, since simple task of merely forming the hanging drop by causing the cell aggregate to be encapsulated in a liquid drop of the medium solution and causing the promoting factor to act on the hanging drop is performed, the manufacture of the sample can be automated. Consequently, a sample that enables a cell aggregate to undergo high-resolution observation and imaging using a microscope can be easily manufactured, and the manufacture of the sample can be easily automated.

A second aspect of the present invention provides a sample manufacturing method that includes: a step of forming a hanging drop consisting of a liquid drop of a culture medium in a hanging state while causing at least one cell to be encapsulated in the liquid drop of the culture medium; a step of culturing the cell inside the hanging drop until a desired cell aggregate is formed; a step of adding to the hanging drop a medium solution that becomes substantially transparent upon gelling or solidifying; and a step of causing the hanging drop to gel or solidify by causing a promoting factor that promotes gelling or solidification of the medium solution to act on the hanging drop.

According to the second aspect of the present invention, a cell aggregate is formed by culturing a cell inside a hanging drop formed of a liquid drop of a culture medium, and consequently there is no need to move the cultured cell aggregate, throughput can be improved, and screening can be performed at a low cost.

A third aspect of the present invention provides a sample manufacturing method that includes: a step of forming a hanging drop consisting of a liquid drop of a culture medium and a medium solution in a hanging state while causing at least one cell to be encapsulated in the liquid drop of the culture medium and the medium solution, the medium solution becoming substantially transparent upon gelling or solidifying; a step of culturing the cell inside the hanging drop until a desired cell aggregate is formed; and a step of causing the hanging drop to gel or solidify by causing a promoting factor that promotes gelling or solidification of the medium solution to act on the hanging drop.

According to the third aspect of the present invention, a hanging drop is formed by injecting a medium solution together with a culture medium, and therefore a step of adding a medium solution after forming the hanging drop can be omitted. Thus, the task can be simplified.

The above-described second aspect may include a step of sucking the culture medium from the hanging drop after culturing the cell and prior to adding the medium solution to the hanging drop.

With this configuration, the hanging drop can be caused to gel or solidify with certainty in the case where the culture medium includes a component that inhibits gelling or solidification of the medium solution.

The above-described aspect may include a step of adding to the medium solution an inhibiting solution, which retards gelling or solidification of the medium solution by inhibiting promotion of gelling or solidification of the medium solution, prior to causing the hanging drop to gel or solidify.

With this configuration, the hanging drop can be caused to take a longer time to gel or solidify. Therefore, provided that the specific gravity of the medium solution constituting the hanging drop is lower than the specific gravity of the cell aggregate, the hanging drop can be caused to gel or solidify in a state where the cell aggregate is located in the vicinity of the lowermost point in the hanging drop due to gravity, and the position of the cell aggregate G in the vertical direction inside the hanging drop can be made to be substantially fixed. In addition, since the cell aggregate ultimately falls along the boundary of the hanging drop, the position of the cell aggregate with respect to the horizontal direction can also be made substantially fixed.

The above-described aspect may include a step of adding to the hanging drop a transparency-inducing solution, which causes the cell aggregate to turn transparent, prior to causing the promoting factor to act on the hanging drop.

With this configuration, illumination light used for performing observation can readily reach the inside of the cell aggregate even in the case of a large cell aggregate. Thus, the internal structure of the cell aggregate can be easily observed regardless of the size of the cell aggregate.

In the above-described aspect, the hanging drop may be formed of a solution having a lower specific gravity than the cell aggregate.

With this configuration, the cell aggregate can be made to move under gravity along the boundary of the hanging drop and become arranged at the lowermost point in the hanging drop.

In the above-described aspect, the promoting factor may be temperature.

In this case, the hanging drop can be caused to gel or solidify by performing the simple task of merely managing the temperature of the medium solution.

In this case, the medium solution may be agarose.

In the above-described aspect, the promoting factor may be light.

In this case, the hanging drop can be caused to gel or solidify by performing the simple task of merely irradiating the medium solution with specific light.

In this case, the medium solution may be an ultraviolet-light-curable liquid resin.

In the above-described aspect, the hanging drop may be caused to gel or solidify by making the medium solution contact a promoting solution serving as the promoting factor.

In this case, the hanging drop can be caused to gel or solidify by performing the simple task of merely causing the medium solution to contact a promoting solution.

In the above-described aspect, the hanging drop may be caused to gel or solidify by being immersed in the promoting solution.

In this case, there is no need for the promoting solution to be injected into the hanging drop, and a plurality of hanging drops can be caused to gel or solidify all at once by being made to contact the promoting solution.

In the above-described aspect, the medium solution may be a sodium alginate solution and the promoting solution may be a calcium solution obtained by dissolving calcium ions.

In the above-described aspect, the medium solution may be an epoxy-based liquid resin, and the promoting solution may be a polyamine solution obtained by dissolving polyamines.

In the above-described aspect, the hanging drop may be formed by using a hanging-drop forming implement that includes a recessed part into which a solution is injected, a hanging-drop forming section that holds a liquid drop of the solution injected into the recessed part in a hanging state while causing the cell aggregate to be encapsulated inside the liquid drop, and a conduit that connects the recessed part and the hanging-drop forming section to each other.

With this configuration, a hanging drop consisting of a liquid drop of a solution in a hanging state is formed as a result of the solution injected into the recessed part of the hanging-drop forming implement moving into the hanging-drop forming section via the conduit. Therefore, a hanging drop can be formed using a simple method of merely injecting a solution into a recessed part.

In the above-described aspect, the solution may be dispensed via the recessed part of the hanging-drop forming implement.

In the above-described aspect, the hanging-drop forming implement may be a multiwell plate having an array structure.

With this configuration, a plurality of hanging drops corresponding to the number of wells can be formed all at once.

A fourth aspect of the present invention provides a sample manufacturing kit that includes: a hanging-drop forming implement having a recessed part into which a solution is injected, a hanging-drop forming section that holds a liquid drop of the solution injected into the recessed part in a hanging state while causing a cell aggregate to be encapsulated inside the liquid drop, and a conduit that connects the recessed part and the hanging-drop forming section to each other; a medium solution that is injected into the recessed part together with a cell and becomes substantially transparent upon gelling or solidifying; and a promoting solution that promotes gelling or solidification of the medium solution.

According to the fourth aspect of the present invention, a hanging drop consisting of a liquid drop of a medium solution in a hanging state is formed as a result of the solution moving into the hanging-drop forming section via the conduit when the medium solution is injected into the recessed part of the hanging-drop forming implement. Then, by making a promoting solution act on the hanging drop in which the cell aggregate is encapsulated to cause the hanging drop to gel or solidify, a sample in which the position of a cell aggregate is fixed inside a substantially transparent hanging drop can be manufactured. Therefore, a sample that enables a cell aggregate to undergo high-resolution observation and imaging using a microscope can be easily manufactured, and the manufacture of the sample can be easily automated.

A fifth aspect of the present invention provides an observation method in which a hanging drop consisting of a liquid drop in a hanging state in which a cell aggregate is encapsulated is held and observation light from the cell aggregate inside the hanging drop is detected.

According to the fifth aspect of the present invention, a cell aggregate can be observed in a state in which the cell aggregate is encapsulated inside the hanging drop. Therefore, the task of dropping hanging drops in wells can be omitted, and a plurality of cell aggregates can be observed in a short period of time.

In the above-described aspect, the gelled or solidified hanging drop in which the cell aggregate is encapsulated may be immersed in a liquid immersion medium that is stored inside a medium container having a transparent part through which light can pass and that has the same refractive index as the solution constituting the hanging drop, and the observation light from the cell aggregate may be detected via the transparent part.

With this configuration, observation light from the cell aggregate is radiated through the transparent part of the medium container without undergoing refraction between the hanging drop and the liquid immersion medium. Therefore, the cell aggregate can be observed at high resolution by detecting observation light from the cell aggregate outside the medium container.

In the above-described aspect, the cell aggregate may be irradiated with illumination light, and the observation light emitted from the cell aggregate may be detected.

With this configuration, desired observation light from the cell aggregate can be generated and observed.

In the above-described aspect, the cell aggregate may be irradiated with the illumination light via the transparent part of the medium container from a direction that intersects a detection optical axis of a detection optical system that detects the observation light emitted from the cell aggregate.

With this configuration, observation light generated over a wide area along a focal plane of the detection optical system can be detected by aligning the focal plane of the detection optical system with the incidence plane of the illumination light.

In the above-described aspect, the cell aggregate may be irradiated with illumination light via the transparent part of the medium container from a direction that intersects a detection optical axis of a detection optical system that detects the observation light emitted from the cell aggregate, and the observation light emitted from the cell aggregate may be detected by the detection optical system.

In the above-described aspect, the cell aggregate may be arranged with a space between the cell aggregate and a bottom surface of the medium container.

If the cell aggregate is in contact with the bottom surface of the medium container, the part of the cell aggregate that is in contact with the bottom surface cannot be satisfactorily illuminated unless the refractive index of the transparent part of the medium container is equal to the refractive index of the liquid immersion medium. With this configuration, the entirety of the cell aggregate can be observed regardless of the refractive index of the transparent part of the medium container.

In the above-described aspect, the cell aggregate may be arranged with a space between the cell aggregate and a bottom surface of the medium container, the cell aggregate may be irradiated with illumination light, and the observation light emitted from the cell aggregate may be detected.

In the above-described aspect, the hanging drop may be held using a hanging-drop forming implement that includes a recessed part into which a solution is injected, a hanging-drop forming section that holds a liquid drop of the solution injected into the recessed part in a hanging state while causing the cell aggregate to be encapsulated inside the liquid drop, and a conduit that connects the recessed part and the hanging-drop forming section to each other.

With this configuration, the hanging drop can be formed and held using a simple method of just injecting a solution into the recessed part, and observation of the cell aggregate can be easily performed.

In the above-described aspect, the solution may be dispensed via the recessed part of the hanging-drop forming implement.

In the above-described aspect, a plurality of the hanging drops having the cell aggregates encapsulated thereinside may be held and made available for observation.

A sixth aspect of the present invention provides an observation device that includes: a hanging-drop forming implement that forms a hanging drop consisting of a liquid drop in a hanging state in which a cell aggregate is encapsulated; a detection optical system that detects observation light emitted from the cell aggregate encapsulated inside the hanging drop formed by the hanging-drop forming implement; and a driving device that changes a relative position of the hanging drop held by the hanging-drop forming implement and a detection position of the detection optical system.

According to the sixth aspect of the present invention, the cell aggregate can be observed by detecting observation light from the cell aggregate inside the hanging drop using the detection optical system by adjusting the relative position of the hanging drop and the detection position of the detection optical system using the driving device in a state where the hanging drop in which the cell aggregate is encapsulated is held by the hanging-drop forming implement. Therefore, a plurality of samples can be observed in a short period of time and at low cost without the use of wells into which hanging drops are dropped.

In the above-described aspect, the hanging-drop forming implement may include a recessed part into which a solution is injected, a hanging-drop forming section that holds a liquid drop of the solution injected into the recessed part in a hanging state while causing the cell aggregate to be encapsulated inside the liquid drop, and a conduit that connects the recessed part and the hanging-drop forming section to each other.

In the above-described aspect, the observation device may further include a medium container in which a liquid immersion medium having the same refractive index as the liquid drop constituting the hanging drop is stored and through which the observation light from the cell aggregate can pass. The driving device may move the hanging-drop forming implement so as to immerse the gelled or solidified hanging drop, in which the cell aggregate is encapsulated, in the liquid immersion medium.

With this configuration, the hanging drop can be immersed in the liquid immersion medium in the medium container by moving the hanging-drop forming implement using the driving device, and as a result, observation light from the cell aggregate encapsulated in the hanging drop can be detected by the detection optical system after passing through the liquid immersion medium and the medium container. Therefore, tomographic images of the cell aggregate that intersect the detection optical axis can be acquired by moving the hanging drop in a direction along the detection optical axis of the detection optical system using the driving device.

In the above-described aspect, the medium container may have an observation-light-transmitting transparent part through which the observation light from the cell aggregate can pass.

With this configuration, the cell aggregate can be observed at high resolution by detecting the observation light from the cell aggregate using the detection optical system after the observation light has passed through the observation-light-transmitting transparent part of the medium container.

In the above-described aspect, the medium container may be held by an objective lens of the detection optical system.

With this configuration, there is no need for a member for specially holding the medium container, and the configuration can be simplified.

In the above-described aspect, the objective lens may be a liquid-immersion objective lens arranged such that an optical axis thereof faces substantially vertically upward, and the medium container may be formed of a leading end of the liquid-immersion objective lens and a cylindrical member one end of which in an axial direction is attached to the leading end.

With this configuration, a medium container can be formed at low cost by using the leading end of the liquid-immersed objective lens as the bottom part of the medium container.

In the above-described aspect, the observation device may include an illumination optical system that irradiates the cell aggregate with illumination light, and the medium container may have an illumination-light-transmitting transparent part through which the illumination light from the illumination optical system passes.

With this configuration, the illumination optical system is arranged outside the medium container, and the cell aggregate can be irradiated with illumination light from outside the medium container via the illumination-light-transmitting transparent part.

In the above-described aspect, the illumination optical system may radiate illumination light having a width and a thickness in directions that intersect an illumination optical axis from a direction that intersects a detection optical axis of the detection optical system.

With this configuration, observation light generated over a wide area along the focal plane in the cell aggregate can be detected by the detection optical system all at once by aligning the focal plane of the detection optical system with the incidence area of the illumination light in the cell aggregate.

In the above-described aspect, an optical axis of the illumination optical system and an optical axis of the detection optical system may be perpendicular to each other.

In the above-described aspect, the illumination optical system may make planar illumination light collected in a direction along a detection optical axis within a field of view of the detection optical system be incident on the cell aggregate.

With this configuration, it is possible to configure a light-sheet microscope that can acquire images of a higher resolution by aligning a focal plane of the detection optical system with an incidence plane of the illumination light in the cell aggregate and detecting observation light generated over a wide area along the focal plane of the detection optical system all at once using the detection optical system.

In the above-described aspect, the illumination optical system may form illumination light having a width in directions that intersect the illumination optical axis by using optical scanning.

In the above-described aspect, the detection optical system may include a microlens that is arranged substantially at an image-forming position and a camera that is arranged subsequent to the microlens.

With this configuration, the focal plane of the detection optical system is aligned with the incidence area of the illumination light in the cell aggregate, and as a result observation light generated over a wide area along the focal plane in the cell aggregate is projected by the microlens and the projected image is captured by the camera. Therefore, a light-field microscope can be configured that can acquire a plurality of sets of image information having different parallaxes in one go.

In the above-described aspect, the hanging-drop forming implement may be a multiwell plate having an array structure that can hold a plurality of the hanging drops. The driving device may sequentially align each cell aggregate with the detection optical axis of the detection optical system by changing the relative position of the hanging drop and the detection position of the detection optical system.

With this configuration, a plurality of cell aggregates can be observed by sequentially aligning the cell aggregates on the detection optical axis of the detection optical system using the driving device.

In the above-described aspect, the liquid immersion medium may include a culture medium, and the liquid drop that constitutes the hanging drop may be composed of a substance through which a culture component of the culture medium can pass.

With this configuration, a plurality of cells encapsulated in a hanging drop can be observed in vivo.

In the above-described aspect, the hanging-drop forming implement may be a multiwell plate having an array structure that can hold a plurality of the hanging drops. The liquid immersion medium may include a culture medium, and the liquid drop that constitutes the hanging drop may be composed of a substance through which a culture component of the culture medium can pass. The driving device may sequentially align each cell aggregate with the detection optical axis of the detection optical system by changing the relative position of the hanging drop and the detection position of the detection optical system.

The above-described aspect may include a control unit that executes sequential control in which time lapse observation is performed.

With this configuration, chronological changes of a plurality of cells encapsulated in a hanging drop can be observed using the control unit.

The sample manufacturing method and sample manufacturing kit according to the present invention afford the advantages that a sample in which a cell aggregate can be observed and imaged at high resolution using a microscope can be easily manufactured, and the manufacture of the sample can be easily automated. In addition, the observation method and observation device according to the present invention afford the advantage that a cell aggregate of a sample manufactured using the sample manufacturing method and sample manufacturing kit can be effectively observed.

### REFERENCE SIGNS LIST

1 hanging-drop forming implement
3 recessed part
5 hanging-drop forming section
7 conduit
13 rod (hanging-drop forming implement)
15 medium container
15a bottom part (transparent part, observation-light-transmitting transparent part)
15b side wall part (transparent part, illumination-light−
transmitting transparent part)
21, 41, 61 observation device
23 detection optical system
25 driving device
27 controller (control unit)
29 objective lens
35 camera
43 illumination optical system
63 microlens array
63a microlens
67 liquid-immersion objective lens
67a leading end
69 cylindrical member
C culture medium
D hanging drop
G cell aggregate
H promoting solution
M medium solution
S cell
T transparency-inducing solution
W liquid immersion medium

1. A sample manufacturing method comprising: a step of forming a hanging drop consisting of a liquid drop of a medium solution in a hanging state while causing at least one cell aggregate to be encapsulated in the liquid drop of the medium solution, the medium solution becoming substantially transparent upon gelling or solidifying; and
a step of causing the hanging drop to gel or solidify by causing a promoting factor that promotes gelling or solidification of the medium solution to act on the hanging drop.

2. A sample manufacturing method comprising: a step of forming a hanging drop consisting of a liquid drop of a culture medium in a hanging state while causing at least one cell to be encapsulated in the liquid drop of the culture medium;
a step of culturing the cell inside the hanging drop until a desired cell aggregate is formed;
a step of adding to the hanging drop a medium solution that becomes substantially transparent upon gelling or solidifying; and
a step of causing the hanging drop to gel or solidify by causing a promoting factor that promotes gelling or solidification of the medium solution to act on the hanging drop.

3. A sample manufacturing method comprising: a step of forming a hanging drop consisting of a liquid drop of a culture medium and a medium solution in a hanging state while causing at least one cell to be encapsulated in the liquid drop of the culture medium and the medium solution, the medium solution becoming substantially transparent upon gelling or solidifying;
a step of culturing the cell inside the hanging drop until a desired cell aggregate is formed; and
a step of causing the hanging drop to gel or solidify by causing a promoting factor that promotes gelling or solidification of the medium solution to act on the hanging drop.

4. The sample manufacturing method according to claim 2, further comprising a step of sucking the culture medium from the hanging drop after culturing the cell and prior to adding the medium solution to the hanging drop.

5. The sample manufacturing method according to claim 1, further comprising: a step of adding to the medium solution an inhibiting solution, which retards gelling or solidification of the medium solution by inhibiting promotion of gelling or solidification of the medium solution, prior to causing the hanging drop to gel or solidify.

6. The sample manufacturing method according to claim 1, further comprising: a step of adding to the hanging drop a transparency-inducing solution, which causes the cell aggregate to turn transparent, prior to causing the promoting factor to act on the hanging drop.

7. The sample manufacturing method according to claim 1, wherein the hanging drop is formed of a solution having a lower specific gravity than the cell aggregate.

8. The sample manufacturing method according to claim 1, wherein the promoting factor is temperature.

9. The sample manufacturing method according to claim 1, wherein the promoting factor is light.

10. The sample manufacturing method according to claim 1, wherein the hanging drop is caused to gel or solidify by making the medium solution contact a promoting solution serving as the promoting factor.

11. The sample manufacturing method according to claim 10, wherein the hanging drop is caused to gel or solidify by being immersed in the promoting solution.

12. The sample manufacturing method according to claim 1, wherein the hanging drop is formed by using a hanging-drop forming implement that includes a recessed part into which a solution is injected, a hanging-drop forming section that holds a liquid drop of the solution injected into the recessed part in a hanging state while causing the cell aggregate to be encapsulated inside the liquid drop, and a conduit that connects the recessed part and the hanging-drop forming section to each other.

13. The sample manufacturing method according to claim 12, wherein the solution is dispensed via the recessed part of the hanging-drop forming implement.

14. A sample manufacturing kit comprising: a hanging-drop forming implement that includes a recessed part into which a solution is injected, a hanging-drop forming section that holds a liquid drop of the solution injected into the recessed part in a hanging state while causing a cell aggregate to be encapsulated inside the liquid drop, and a conduit that connects the recessed part and the hanging-drop forming section to each other; a medium solution that is injected into the recessed part together with a cell; and a promoting solution that promotes gelling or solidification of the medium solution.

15. An observation method, wherein a hanging drop consisting of a liquid drop in a hanging state in which a cell aggregate is encapsulated is held, and observation light from the cell aggregate inside the hanging drop is detected.

16. The observation method according to claim 15, wherein the gelled or solidified hanging drop in which the cell aggregate is encapsulated is immersed in a liquid immersion medium that is stored inside a medium container having a transparent part through which light can pass, and the observation light from the cell aggregate is detected via the transparent part.

17. The observation method according to claim 15, wherein the cell aggregate is irradiated with illumination light and the observation light emitted from the cell aggregate is detected.

18. The observation method according to claim 16, wherein the cell aggregate is irradiated with illumination light via the transparent part of the medium container from a direction that intersects a detection optical axis of a detection optical system that detects the observation light emitted from the cell aggregate.

19. The observation method according to claim 16, wherein the cell aggregate is arranged with a space between the cell aggregate and a bottom surface of the medium container, the cell aggregate is irradiated with illumination light, and the observation light emitted from the cell aggregate is detected.

20. The observation method according to claim 15, wherein the hanging drop is held using a hanging-drop forming implement that includes a recessed part into which a solution is injected, a hanging-drop forming section that holds a liquid drop of the solution injected into the recessed part in a hanging state while causing the cell aggregate to be encapsulated inside the liquid drop, and a conduit that connects the recessed part and the hanging-drop forming section to each other.

21. The observation method according to claim 20, wherein the solution is dispensed via the recessed part of the hanging-drop forming implement.

22. An observation device comprising: a hanging-drop forming implement that forms a hanging drop consisting of a liquid drop in a hanging state in which a cell aggregate is encapsulated; a detection optical system that detects observation light emitted from the cell aggregate encapsulated inside the hanging drop formed by the hanging-drop forming implement; and a driving device that changes a relative position of the hanging drop held by the hanging-drop forming implement and a detection position of the detection optical system.

23. The observation device according to claim 22, wherein the hanging-drop forming implement includes a recessed part into which a solution is injected, a hanging-drop forming section that holds a liquid drop of the solution injected into the recessed part in a hanging state while causing the cell aggregate to be encapsulated inside the liquid drop, and a conduit that connects the recessed part and the hanging-drop forming section to each other.

24. The observation device according to claim 22, further comprising: a medium container in which a liquid immersion medium is stored and through which the observation light from the cell aggregate can pass; wherein the driving device moves the hanging-drop forming implement so as to immerse the gelled or solidified hanging drop, in which the cell aggregate is encapsulated, in the liquid immersion medium.

25. The observation device according to claim 24, wherein the medium container has an observation-light-transmitting transparent part through which the observation light from the cell aggregate can pass.

26. The observation device according to claim 25, wherein the medium container is held by an objective lens of the detection optical system.

27. The observation device according to claim 24, further comprising: an illumination optical system that irradiates the cell aggregate with illumination light, wherein the medium container has an illumination-light-transmitting transparent part through which the illumination light from the illumination optical system passes.

28. The observation device according to claim 27, wherein the illumination optical system radiates illumination light having a width and a thickness in directions that intersect an illumination optical axis from a direction that intersects a detection optical axis of the detection optical system.

29. The observation device according to claim 27, wherein the illumination optical system makes planar illumination light collected in a direction along a detection optical axis within a field of view of the detection optical system be incident on the cell aggregate.

30. The observation device according to claim 27, wherein the detection optical system includes a microlens that is arranged substantially at an image-forming position and a camera that is arranged subsequent to the microlens.

31. The observation device according to claim 22, wherein the hanging-drop forming implement is a multiwell plate having an array structure that can hold a plurality of the hanging drops, and the driving device sequentially aligns each cell aggregate with the detection optical axis of the detection optical system by changing the relative position of the hanging drop and the detection position of the detection optical system.

32. The observation device according to claim 24, wherein the liquid immersion medium includes a culture medium, and the gelled or solidified hanging drop in which the cell aggregate is encapsulated is composed of a substance through which a culture component of the culture medium can pass.

33. The sample manufacturing method according to claim 2, further comprising: a step of adding to the medium solution an inhibiting solution, which retards gelling or solidification of the medium solution by inhibiting promotion of gelling or solidification of the medium solution, prior to causing the hanging drop to gel or solidify.

34. The sample manufacturing method according to claim 2, further comprising: a step of adding to the hanging drop a transparency-inducing solution, which causes the cell aggregate to turn transparent, prior to causing the promoting factor to act on the hanging drop.

35. The sample manufacturing method according to claim 2, wherein the hanging drop is formed of a solution having a lower specific gravity than the cell aggregate.

36. The sample manufacturing method according to claim 2, wherein the promoting factor is temperature.

37. The sample manufacturing method according to claim 2, wherein the promoting factor is light.

38. The sample manufacturing method according to claim 2, wherein the hanging drop is caused to gel or solidify by making the medium solution contact a promoting solution serving as the promoting factor.

39. The sample manufacturing method according to claim 38, wherein the hanging drop is caused to gel or solidify by being immersed in the promoting solution.

40. The sample manufacturing method according to claim 2, wherein the hanging drop is formed by using a hanging-drop forming implement that includes a recessed part into which a solution is injected, a hanging-drop forming section that holds a liquid drop of the solution injected into the recessed part in a hanging state while causing the cell aggregate to be encapsulated inside the liquid drop, and a conduit that connects the recessed part and the hanging-drop forming section to each other.

41. The sample manufacturing method according to claim 40, wherein the solution is dispensed via the recessed part of the hanging-drop forming implement.

42. The sample manufacturing method according to claim 3, further comprising: a step of adding to the medium solution an inhibiting solution, which retards gelling or solidification of the medium solution by inhibiting promotion of gelling or solidification of the medium solution, prior to causing the hanging drop to gel or solidify.

43. The sample manufacturing method according to claim 3, further comprising: a step of adding to the hanging drop a transparency-inducing solution, which causes the cell aggregate to turn transparent, prior to causing the promoting factor to act on the hanging drop.

44. The sample manufacturing method according to claim 3, wherein the hanging drop is formed of a solution having a lower specific gravity than the cell aggregate.

45. The sample manufacturing method according to claim 3, wherein the promoting factor is temperature.

46. The sample manufacturing method according to claim 3, wherein the promoting factor is light.

47. The sample manufacturing method according to claim 3, wherein the hanging drop is caused to gel or solidify by making the medium solution contact a promoting solution serving as the promoting factor.

48. The sample manufacturing method according to claim 47, wherein the hanging drop is caused to gel or solidify by being immersed in the promoting solution.

49. The sample manufacturing method according to claim 3, wherein the hanging drop is formed by using a hanging-drop forming implement that includes a recessed part into which a solution is injected, a hanging-drop forming section that holds a liquid drop of the solution injected into the recessed part in a hanging state while causing the cell aggregate to be encapsulated inside the liquid drop, and a conduit that connects the recessed part and the hanging-drop forming section to each other.

50. The sample manufacturing method according to claim 49, wherein the solution is dispensed via the recessed part of the hanging-drop forming implement.
